(19) United States
(12) Patent Application Publication
(10) Pub. No.: US 2023/0109581 A1
(43) Pub. Date: Apr. 6, 2023

(54) COLOR READING FOR DIAGNOSTIC TESTS

(71) Applicant: Detect, Inc., Guilford, CT (US)

(72) Inventors: Jonathan M. Rothberg, Miami Beach, FL (US); Jose Camara, Saratoga, CA (US); Alexander B. Leffell, New Haven, CT (US)

(21) Appl. No.: 17/958,265

(22) Filed: Sep. 30, 2022

Related U.S. Application Data
(60) Provisional application No. 63/251,300, filed on Oct. 1, 2021.

(51) Int. Cl. G01N 33/569; C12Q 1/70; G01N 33/487; G01N 33/543; G16B 50/30; G16H 10/40

(52) U.S. Cl. CPC G01N 33/56983; C12Q 1/70; G01N 33/48792; G01N 33/54386; G16B 50/30; G16H 10/40; G01N 2333/165; G01N 33/54388

(57) ABSTRACT

Provided herein, in some embodiments, are rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. In one embodiment, a rapid test method is provided comprising performing an isothermal nucleic acid amplification-based rapid test, accessing fluorescence data of a reaction tube of the test, and visually detecting, via the fluorescence data, presence or absence of a target pathogen, such as COVID-19 and/or an influenza virus and/or a target nucleic acid.

### RELATED APPLICATION

This application claims the benefit under 35 U.S.C. 119(e) of U.S. provisional application Ser. No. 63/251,300 filed Oct. 1, 2021, which is incorporated by reference herein in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Provided herein are a number of diagnostic tests useful for detecting target nucleic acid sequences. The tests, as described herein, are able to be performed in a point-of-care (POC) setting or home setting without specialized equipment.

Therefore, in some aspects, the disclosure provides a rapid test method comprising performing an isothermal nucleic acid amplification-based rapid test, accessing fluorescence data of a reaction tube of the test, and visually detecting, via the fluorescence data, presence or absence of COVID-19 and/or an influenza virus and/or a target nucleic acid.

In some aspects, the disclosure provides an apparatus comprising at least one computer hardware processor and at least one non-transitory computer-readable storage medium storing processor executable instructions that, when executed by the at least one computer hardware processor, cause the at least one computer hardware processor to perform accessing fluorescence data of a reaction tube of a test, and visually detecting, via the fluorescence data, presence or absence of COVID-19 and/or an influenza virus and/or a target nucleic acid.

In some aspects, the disclosure provides a non-transitory computer-readable media comprising instructions that, when executed by one or more processors on a computing device, are operable to cause the one or more processors to visually detect, via fluorescence data of a reaction tube, presence or absence of COVID-19 and/or an influenza virus and/or a target nucleic acid.

### BRIEF DESCRIPTION OF THE DRAWINGS

FIG. 1 is a diagram showing an exemplary reaction tube and a visual test result detection module, according to some embodiments;

FIG. 2 is a diagram showing a visual test result detection module with a light source array and a light detector array, according to some embodiments;

FIG. 3 is a flow chart showing a computerized method for visually determining a test reading or a test result, according to some embodiments;

FIG. 4 is a diagram showing excitation illumination and detection data from a light detector, according to some embodiments;

FIG. 5 is a diagram showing a plurality of excitation illuminations and detection data from a plurality of light detectors, according to some embodiments;

FIG. 6 is a diagram showing an apparatus for visually detecting, via a reaction tube, presence or absence of a target (e.g., COVID-19 and/or an influenza virus and/or a target nucleic acid and/or other target pathogen), according to some embodiments;

FIG. 7 shows diagnostic kits comprising a sample-collecting component, a reaction tube, a detection component, and a temperature control device, according to some embodiments;

FIG. 8 shows, according to some embodiments, a cartridge comprising a first reservoir, a second reservoir, a third reservoir, a vent path, a detection region, and a pumping tool;

FIG. 9 shows, according to some embodiments, a diagnostic kit comprising a sample-collecting component and a cartridge; and

FIG. 10 shows, according to some embodiments, a diagnostic device comprising a plurality of blister packs.

### DETAILED DESCRIPTION

The present disclosure provides diagnostic devices, systems, and methods for rapidly and in a home environment visually detecting one or more target nucleic acid sequences (e.g., a nucleic acid sequence of a pathogen, such as SARS-CoV-2 or an influenza virus). A diagnostic system, as described herein, may be self-administrable and comprise a sample-collecting component (e.g., a swab) and a diagnostic device. In some embodiments, the diagnostic system may comprise one or more consumables (e.g., a test tube, a test tube cap, a swab, a card, a label) which may be discarded after use or configured for multiple uses with the diagnostic device. The diagnostic device may comprise a reaction tube, a cartridge, and/or a blister pack, according to some embodiments. The diagnostic device includes a visual test result detection component that can visually determine a test result. In some cases, the diagnostic device comprises an additional detection component (e.g., colorimetric assay), results of which are self-readable, or automatically read by the visual test result detection component and/or a separate computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may comprise an integrated temperature control device (e.g., a heater, a cooling device, or any other suitable temperature control device), or the diagnostic system may comprise a separate temperature control device (e.g., a heater, a cooling device, or any other suitable temperature control device). The isothermal amplification technique employed in some embodiments yields not only fast but very accurate results.

### I. Color Reading Techniques

Diagnostic tests often leverage a detection component to provide the test results, such as a lateral flow strip or a colorimetric assay. Typically, the detection component is not used until the end of the test: one or more associated test reactions (e.g., an isothermal reaction) and/or other test chemistry is completed prior to using the detection component. In particular, if a test solution is provided to a detection component too soon, it can result in an invalid test. In order to ensure that the test chemistry is complete prior to using the detection component, the inventors have recognized and appreciated that conventional tests often require that a user wait a predetermine amount of time before exposing a solution to the detection component, regardless of when the chemistry was actually completed. In particular, some diagnostic tests often require a user wait a predetermined amount of time that is typically longer than the amount of time required to complete the reactions to ensure that the chemistry is complete. It is therefore not uncommon for test steps to require a user to wait a half hour, an hour, etc., prior to providing the solution to the detection component, even though the necessary test reactions may be completed much sooner. The inventors have therefore appreciated that such approaches can unnecessarily increase the amount of time required to complete a test, which can particularly be an issue for rapid tests (e.g., where it is desirable to know the test result as soon as possible).

The inventors have further appreciated that using such detection components often requires that the user perform one or more test steps to expose the solution to the diagnostic component. For example, a test component containing the solution (e.g., a reaction tube) may need to be opened to expose the liquid to the detection component. This can provide a contamination risk to the user, introduce environmental contaminants to the liquid that can affect the test result, and/or the like. As another example, the test component may need to be placed into a device that houses the detection component. For example, a reaction tube may be placed into a chimney-shaped component designed to receive the reaction tube. In order to expose the solution to a detection component of the chimney-shaped component, the user may be required to perform one or more further actions, such as pressing down on the reaction tube in order to puncture a portion of the reaction tube to allow the liquid to flow to the detection component. Such steps may require the user to exert a sufficient amount of force that can be difficult for some users to exert.

The inventors have further recognized and appreciated that interpreting the visual information indicated on the detection component of a diagnostic test can present further challenges. In general, with conventional techniques, a human observer (e.g., a doctor, nurse, or other medical professional) may determine the results of the diagnostic test based on the visual information of the diagnostic test detection component. Human error in interpreting the visual information indicated on the detection component of the diagnostic test can lead to errors in determining the test results. For example, a user of a diagnostic test may misread the test as positive when it in fact it is negative or invalid.

A further challenge recognized and appreciated by the inventors is that the user of the diagnostic test may be unable to read the test result and/or may incorrectly determine the test result. For example, in some cases, the diagnostic test user may be an individual without medical training (e.g., an individual who is not a nurse, doctor, or other expert) and/or an individual without sufficient training with the diagnostic test (e.g., a clinician, including a nurse, doctor, or other expert, who is not familiar with the diagnostic test). This may occur, for example, in the context of self-administered or at-home diagnostic tests, which may be carried out without the presence of a medical professional. Without medical training, the user of the diagnostic test may be unable to interpret the visual information indicated on the detection component, or may do so with decreased accuracy, confidence, and/or speed relative to a medical professional. Additionally or alternatively, this can occur in the clinical setting, when a clinician administers the test but is unable to interpret visual information indicated on the detection component (e.g., due to insufficient training of the clinician, etc.). Users may be unable to read the test for various other reasons, such as users that may have vision problems, poor lighting and/or other environmental conditions (e.g., direct sunlight if administered outdoors), intellectual issues, unintentional confusion, and so on.

The inventors have further recognized and appreciated that, in some cases, the visual information indicated on the detection component of the diagnostic may be less visible or clear than desired (e.g., lines on a test strip may be faint or blurred, the colors of the detection component may be difficult to distinguish). In some cases, it may be difficult or impossible for a human to perceive some or all of the visual information indicated on the diagnostic test detection component, resulting in reduced the accuracy of the corresponding test results. For example, in the case of a lateral flow control test strip, a user may mistakenly identify the test results as a false negative if a line indicating a positive result is faded, blurred, or otherwise difficult to perceive.

Recognizing the foregoing, the inventors have developed a diagnostic system that can visually determine a test reading and/or test result in real time during the test procedure (e.g., without needing to use a lateral flow assay (LFA) component). In some embodiments, the techniques include performing a rapid test, and visually detecting the test reading or result using the actual test components (e.g., components used to perform the test chemistry). For example, an isothermal nucleic acid amplification-based rapid test can be performed using a reaction tube, and the techniques can visually detect, via the reaction tube, the presence or absence of COVID-19 and/or an influenza virus and/or a target nucleic acid. The techniques can therefore visually monitor the test component (e.g., a reaction tube, blister pack, sample well) in real time while performing the rapid test, rather than waiting until the end of the reaction and/or waiting a predetermined time period. For example, the techniques can visually monitor an isothermal amplification process until a sufficient determination can be made as to the result of the test.

In some embodiments, the visual test result detection is performed using a device that includes one or more illumination sources for illuminating a test component of the rapid test (e.g., a reaction tube), and one or more detectors for imaging the test component. The test component can include, for example, one or more probes (e.g., fluorescent probes, such as single quenched probes, double quenched probes, etc.) that can be used to monitor for one or more test events. In some embodiments, the test component includes one probe. In some embodiments, the test component includes a plurality of probes (e.g., probes of different colors and/or fluorescence). Multiple probes can be used to monitor for multiple test results. For example, multiple probes can be used to monitor for multiple of COVID-19, an influenza virus, and/or a target nucleic acid. As an example, three probes can be used, with one probe for each of COVID-19, an influenza virus, and a target nucleic acid. As a further example, four probes can be used, with one probe for each of COVID-19, an influenza virus, and a target nucleic acid, and one probe for a control (e.g., to monitor for an invalid test). As another example, four probes can be used, with two probes for COVID-19 (e.g., each probe for a different COVID-19 gene), and one probe for each of an influenza virus and a target nucleic acid. For example, two different quenched probes can be used to detect two different genes associated with COVID-19 (e.g., such that the ratio of the two different genes can provide information on the stage of the COVID-19 infection). In some embodiments, separate test components (e.g., reaction tube and/or sample wells) can be used for different probes (e.g., rather than having a single component include all of the probes).

In some embodiments, the visual test result detection techniques described herein can be provided as a part of an existing rapid test kit or as part of a test kit component. For example, if a test kit includes a temperature control device (e.g., a heating device and/or cooling device) configured to receive a reaction tube in order to control the temperature of a solution contained within a reaction tube to carry out the testing process, then a visual test result detection device can be provided as part of the temperature control device. For example, the visual test result detection device can be configured for use with the temperature control device, or integrated as part of the temperature control device. In some embodiments, the visual test result detection device includes one or more of: an illumination source, a detector, a wireless communication module (e.g., WiFi, Bluetooth, RFID, etc.), at least one processor, and/or one or more computer storage devices (e.g., memory, non-volatile storage, etc.).

The visual detection techniques provided herein can therefore provide for real-time test monitoring and detection that addresses various deficiencies of conventional techniques. For example, the visual detection techniques can be used to visually monitor an assay (e.g., an isothermal amplification process) in real-time during the test process. As a result, the techniques only need to monitor the test process until the point at which the techniques can make a sufficient determination of progress of the test reaction and/or the test result (e.g., by detecting fluorescence emitted by one or more probes). Such techniques therefore do not require waiting any predetermined period(s) of time, as required by some tests (e.g., 30 or 40 minutes, or more). Additionally or alternatively, the techniques provide for monitoring the test components (e.g., the contents of the reaction tube, the sample wells themselves, etc.) in a manner that does not require opening the test components and/or subjecting the test components to force in order to provide the contents to a detection device. Further, by automatically determining the test result from the visual reading and/or monitoring, the test result is not subject to user error (e.g., due to insufficient training or experience, faint visual indicators, etc.).

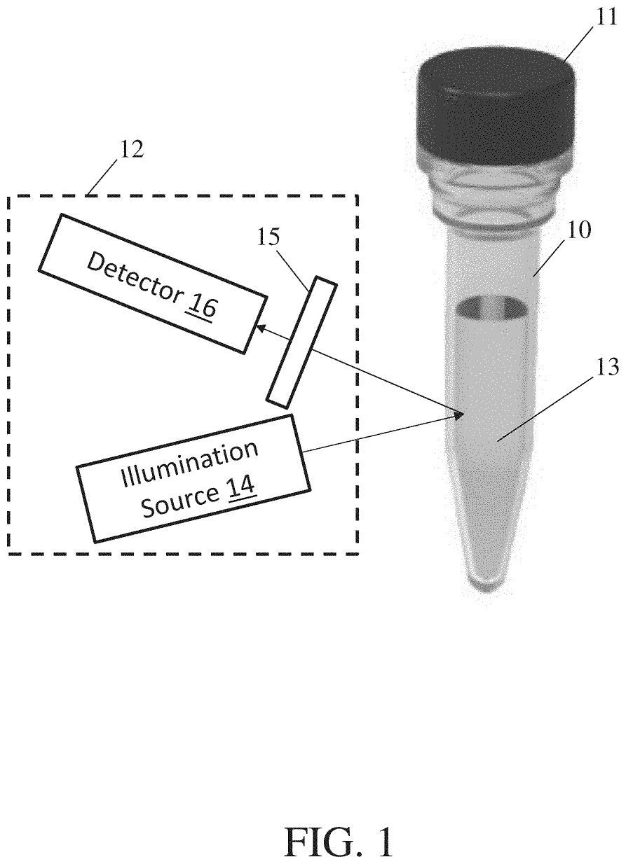

FIG. 1 is a diagram showing an exemplary reaction tube 10 and a visual test result detection module 12, according to some embodiments. The reaction tube 10 has a cap 11 and contains contents 13. In some embodiments, as described herein, the contents 13 of the reaction tube 10 include one or more probes (e.g., quenched probes) to provide information regarding the test. The visual test result detection module 12 includes, in this example, an illumination source 14 and a detector 16, shown in this example separate from the reaction tube 10. The illumination source 14 is configured to illuminate the reaction tube 10 such that light emitted from the illumination source 14 illuminates the contents 13 of the reaction tube 10, which is detected by the detector 16. The visual test result detection module 12 can optionally include one or more optical devices 15, such as an optical filter, lens, etc., through which the detector 16 detects light emitted from the contents 13 of the reaction tube 10.

The illumination source can be any suitable device capable of illuminating the reaction tube (and/or other component(s), as described herein). In some embodiments, the illumination source is configured to provide illumination at one or a plurality of wavelengths (e.g., for excitation diversity). In some embodiments, the illumination source comprises a programmable current illumination source. In some embodiments, the illumination source comprises a constant current illumination source. In some embodiments, the illumination source comprises a light emitting diode (LED). In some embodiments, the illumination source comprises a laser diode. In some embodiments, the laser diode comprises one or more specific wavelengths (e.g., red, blue, etc.).

The light detector can be any suitable device capable of imaging the reaction tube (and/or other component(s), as described herein). In some embodiments, the light detector comprises a photodetector. The photodetector can be, for example, a photodiode (e.g., a silicon photodiode), a camera, and/or the like. In some embodiments, the light detector comprises a transimpedance amplifier. In some embodiments, the light detector can include one or more components to address one or more current aspects of the light detector. For example, in some embodiments the light detector can include reverse biasing to address, at least partially, dark current of the light detector. In some embodiments, as described further herein, the light detector can be operated in a locked-fashion with the illumination sources. In some embodiments, the light detector may include a heat sync and/or other cooling device (e.g., a thermoelectric cooler). For example, a cooling device may be used if the light detector is disposed in a part of the test kit that is subject to heat (e.g., a heater).

In some embodiments, as shown in FIG. 1 for example, the light detector can include a filter, a lens, and/other optical component in the optical path of the light detector. For example, the filter can be part of and/or disposed in the optical path of the light detector (e.g., between the light detector and the reaction tube). In some embodiments, the light detector comprises a plurality of filters. In some embodiments, the filter comprises a glass filter. The glass filter can be, for example, a colored glass filter. In some embodiments, one or more lenses can be disposed in the optical path of the light detector. For example, a lens can be used to essentially expose or map a larger portion of a reaction tube to the comparably smaller area of a photodiode.

In some embodiments, the visual result detection device can include a plurality of optical components. For example, a first optical component can be disposed in the optical path of the illumination source (e.g., a lens to focus the illumination on a component, such as the reaction tube, a filter to filter the color of the illumination, etc.), and a second optical component can be disposed in the optical path of the light detector (e.g., a lens to expose the detector to a larger area of the test component).

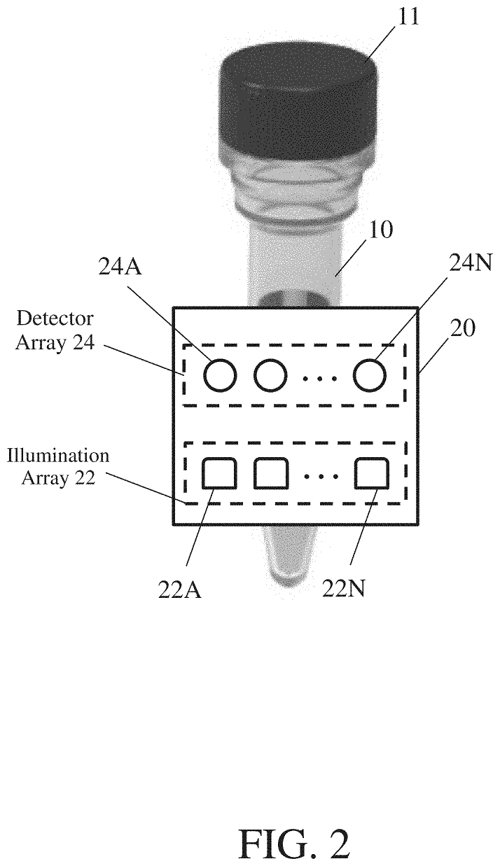

In some embodiments, the visual test result detection module can include a plurality of detectors and/or a plurality of illumination sources. As an illustrative example, FIG. 2 is a diagram showing a visual test result detection module 20 with an illumination array 22 and a light detector array 24, according to some embodiments. The illumination array 22 has a plurality of illumination sources 22A through 22N, and the detector array 24 has a plurality of light detectors 24A through 24N.

In such embodiments, the visual result detection device can include one or more optical components associated with one or more of the plurality of detectors and/or illumination sources. For example, in embodiments with a plurality of illumination sources (e.g., a plurality of photodiodes), the apparatus can include a plurality of optical components that are associated with one or more of the plurality of illumination sources. As an illustrative example, each of a plurality of photodiodes can be optically coupled to an associated filter that is different than filters optically coupled to other photodiodes of the plurality of photodiodes. For example, different filters can be used on different photodetectors to provide for multi-band spectral binning (e.g., if monitoring for a plurality of probes and/or the presence/absence of a single probe). For example, the filters can be used to capture color images (e.g., images of various wavelengths) when using a white light illumination source. The filter can be, for example, a Bayer filter mosaic comprising a plurality of red color filter(s), green color filter(s), and/or blue color filter(s). For example, some embodiments the light detector can include an array of photosensors, and a Bayer filter mosaic can be disposed in the optical path between the array of photosensors and the reaction tube. In some embodiments, the filter can comprise a filter wheel that comprises red color filter(s), green color filter(s), and/or blue color filter(s). For example, the light detector can be a monochrome photosensor, and the wheel can be disposed in an optical path between the monochrome photosensor and the reaction tube.

While the examples discussed in conjunction with FIGS. 1-2 are discussed in the context of detecting color using a reaction tube, it should be appreciated that the color reading techniques described herein can be used to detect any illumination-related aspect (e.g., fluorescence, color, wavelength(s), intensity, lifetime, and/or the like) of any component of a diagnostic system or test component described herein. For example, the component can be a blister pack, a colorimetric assay, a sample well, and/or any other component of the test where the color of the component can be used, at least in part, to determine an aspect of the test (e.g., a test reading and/or a test result). In some embodiments, the component of the diagnostic system may comprise one or more wells. In some embodiments, each well of the diagnostic system may be configured to receive one or more consumables of the diagnostic system. For example, each well of the diagnostic system may be configured to receive a reaction tube or a swab. In some embodiments, multiple wells of the diagnostic system may receive consumables, such as in a simultaneous or staggered manner. As described herein, the visual result detection device may be further configured to monitor and/or detect a color of the contents of well(s), such as to monitor and/or detect fluorescence emitted by one or more quenched probes. In some embodiments, the color of some or all of the wells may be monitored and/or detected together (e.g., with a single visual result detection device for multiple wells). In some embodiments, the color of each well may be monitored and/or detected independently (e.g., with separate visual result detection devices for one or more wells).

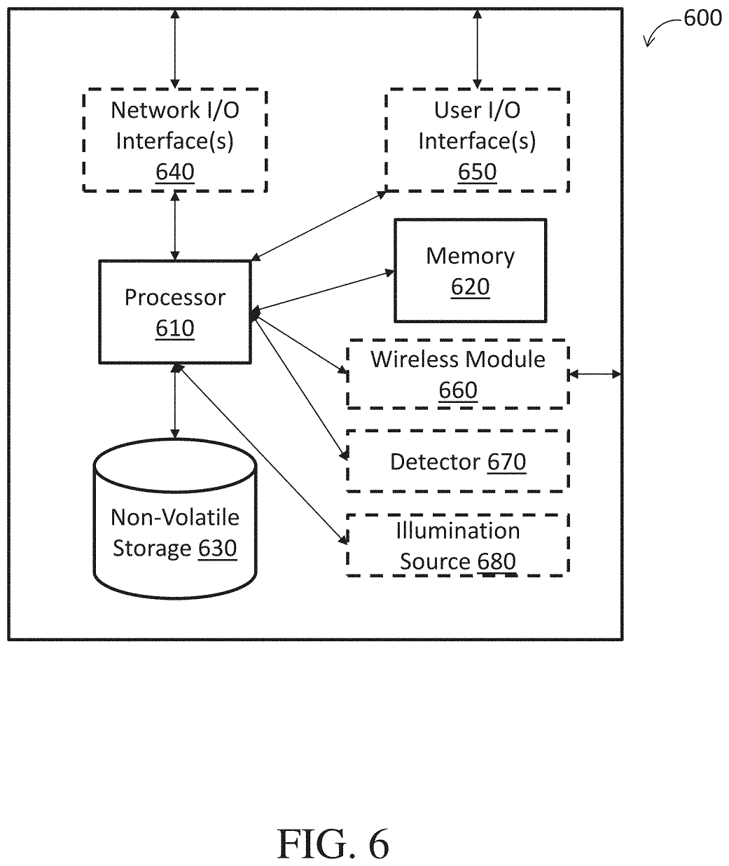

In some embodiments, as discussed in conjunction with FIG. 6, the visual result detection device can be part of an apparatus (e.g., a diagnostic system, a test kit, etc.). The apparatus can include at least one computer hardware processor that interfaces with and/or controls the behavior of the visual result detection device. The processor can control, for example, the detector(s), illumination source(s), and/or one or more other components such as heating or cooling mechanisms, speakers, displays, or any other mechanical or electronic components of the apparatus. In some embodiments, the visual result detection device and the one or more processors can be part of a rapid test diagnostic system.

In some embodiments, the visual result detection device may further comprise one or more receiving components. A receiving component may be, for example, one or more electrical connectors (e.g., a conductive contact point or probe), an RFID reading component (e.g., an antenna or other suitable circuitry for reading RFID tags), or a wireless connection point (e.g., a Bluetooth or WiFi adapter, or any other suitable wireless access circuitry). In some embodiments, a receiving component may be in physical contact or proximity with one or more devices (e.g., a user device, such as a smartphone or other computing device).

In some embodiments, the diagnostic system may comprise a physical encoding of test instructions. For example, the test instructions can configure the visual result detection device to illuminate and/or image the reaction tube and/or other information of interest. As another example, the test instructions can additionally or alternatively configure the visual result detection device to process imaged information of one or more color aspects of the reaction tube to determine a test reading and/or test result. In some embodiments, information encoded in the physical encoding may be accessible using a receiving component of the visual result detection device. According to some embodiments, the physical encoding may comprise, for example, one or more electrical connectors (e.g., a conductive contact point or probe), an RFID tag or an NFC tag (e.g., integrated with or adhered to a cap of a reaction tube, or included as part of the diagnostic device), computer-executable instructions stored in a non-transitory computer storage device (e.g., non-volatile memory, a FLASH drive, etc.), a visual encoding (e.g., a printed data matrix code, such as a barcode, QR code, or any other suitable encoding), and/or the like.

The information of the physical encoding may be accessible using a corresponding receiving component of the visual result detection device in any suitable manner. For example, if the physical encoding comprises one or more electrical connectors, and the receiving component comprises one or more electrical connectors, then the information of the physical encoding may be accessible to the visual result detection device via physical contact between the electrical connectors. As another example, if the physical encoding comprises an RFID tag or NFC tag (e.g., disposed on the reaction tube, a cap of the reaction tube, etc.) and the receiving component comprises an RFID reading component or an NFC reading component, then the information of the physical encoding may be accessible when the RFID or NFC reading component activates the RFID tag or NFC tag comprising the physical encoding. As a further example, if the physical encoding comprises stored computer-executable instructions and the receiving component comprises a wireless communication module (e.g., a Bluetooth, WiFi, or other wireless adapter), then the information of the physical encoding may be accessed when the wireless communication module establishes a connection with a second wireless communication module in communication with the computer-executable instructions, such that the receiving component can wirelessly receive the computer-executable instructions. As another example, if the physical encoding comprises a visual encoding (such as a dot matrix code), then the information of the physical encoding may be accessible by capturing and/or processing an image of the dot matrix code.

Regardless of the nature of the physical encoding and/or the corresponding receiving component, the physical encoding may comprise an encoding of control information (e.g., test information) for the visual result detection device. As described further herein, for example, the visual result detection device can use the control information to determine how to illuminate the test component(s), how to image the test components, and/or how to process images of the test components. The visual result detection device can use the control information to determine, for example, a test reading or a test result, based on the color of one or more components of the test apparatus in the images (e.g., the contents of a reaction tube, sample well, colorimetric assay, and/or the like). For example, the visual result detection device can determine the presence or absence of illumination associated with one or more probes.

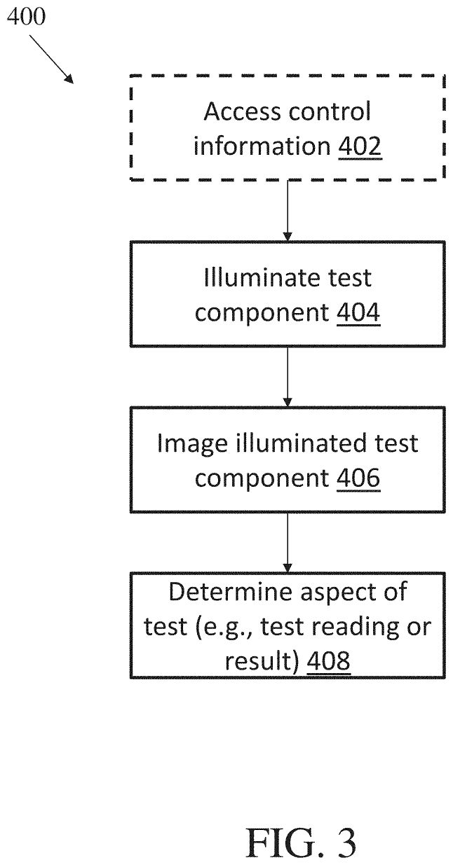

FIG. 3 is a flow chart showing a computerized method 400 for determining a test reading or result, according to some embodiments. The flow chart depicts an illustrative computerized method 400 for using the system of, for example, FIGS. 1-2, according to some embodiments. In some embodiments, method 400 may be carried out by control circuitry of the visual result detection device. In some embodiments, the control circuitry may comprise one or more processors, as described herein at least with respect to FIG. 6.

Method 400 begins at act 402 with receiving, at the visual result detection device, control information of a physical encoding. As indicated by the dashed lines in FIG. 3, act 402 is optional, as in some embodiments the visual result detection device may be pre-programmed with part of and/or all of the control information. As described herein, the control information may be accessible using a receiving component of the visual result detection device. In some embodiments, the control information may be received by the visual result detection device automatically (e.g., when a consumable is received in a well of the test apparatus). In some embodiments, receiving the control information may rely on further user action. For example, if the physical encoding is an RFID tag or NFC tag, the user may be directed to place the component with the tag in contact or proximity with the RFID reading component or NFC reading component of the visual result detection device. This may comprise, for example, touching a location on the visual result detection device with the component (e.g., a marked location on the visual result detection device, which may be in contact or proximity with the RFID tag or NFC tag reading component). If the physical encoding comprises a visual encoding, such as a dot matrix code, then the user may be directed to use the visual result detection device to process the visual encoding. For example, the user may need to capture an image of the visual encoding, and/or direct the electronic device to establish a wireless connection (e.g., a Bluetooth or WiFi connection) with the visual result detection device based on the processed visual encoding.

In some embodiments, the test instructions are customized based on one or more aspects of the test. For example, in some embodiments the test instructions are customized based on contents of the component (e.g., the contents of the reaction tube, the sample well, etc.). In some embodiments, the test instructions are customized based on one or more fluorescent probes and/or dyes contained within the test component. In some embodiments, the test instructions are customized based on a test being performed with the reaction tube. In some embodiments the test instructions are customized to configure the device to detect the presence or absence of COVID-19 and/or the influenza virus and/or the target nucleic acid. In some embodiments, if the visual result detection device and/or the test apparatus is reusable, the visual result detection device can be configured to modify and/or update the test instructions for each test.

Method 400 proceeds to act 404, and the visual result detection device illuminates the test component using the illumination source. The visual result detection device can illuminate the test component at one or a plurality of different times. As described herein, in some embodiments the illumination source comprises a plurality of illumination sources, and the visual result detection device can be configured to illuminate the test component by the plurality of different illumination sources (e.g., at the same and/or different illumination times). For example, different illumination sources may be configured to provide illumination at different wavelengths to detect for the presence of different probes. As a result, it can be desirable to stagger illumination by the multiple illumination sources to provide for discrete sensing opportunities for each probe.

At act 406, the visual result detection device images the illuminated test component. In some embodiments, the visual result detection device can be configured to perform acts 404 and 406 in a coordinated fashion. For example, in some embodiments the test instructions comprise data indicative of light patterns used to detect the presence or absence of one or more fluorescent probes that are indicative of the presence of COVID-19 and/or the influenza virus and/or a target nucleic acid. For example, in some embodiments a test, such as a rapid test (e.g., an isothermal nucleic acid amplification-based rapid test) is performed, and the visual result detection device visually detects, via a reaction tube of the test, the presence or absence of a probe associated with COVID-19 and/or an influenza virus and/or a target nucleic acid. In some embodiments, the test instructions additionally or alternatively comprise data indicative of a light pattern used to detect an invalid test. As a result, the test instructions can configure the visual result detection device to illuminate and image the component(s) accordingly to detect a desired test result (and to optionally detect an invalid test).

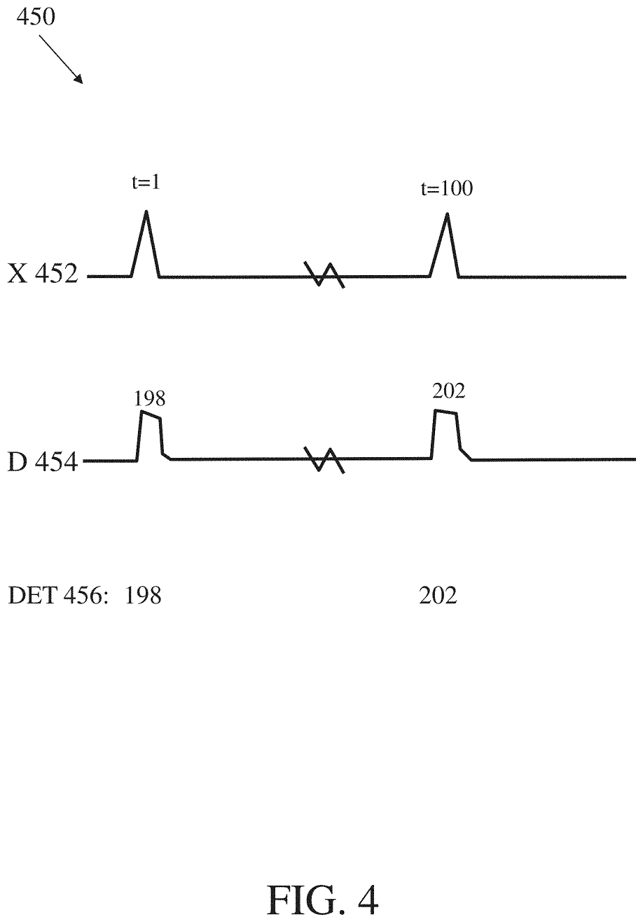

FIG. 4 is a diagram 450 showing excitation illumination 452 and detection data 454 from a light detector, according to some embodiments. As shown, the visual result detection device is configured to illuminate the test component at various times t=1 through t=100, in this example. As a result, the visual result detection device can be configured to illuminate the component at a plurality of illumination periods, and capture imaging data for each illumination period as shown by DET 456. By visually monitoring the test component (e.g., by monitoring for fluorescence associated with a probe), the techniques can monitor for detection changes, such as increases and/or decreases over time. Such time-series data can be used to determine when a sufficient amount of data has been observed in order to determine an associated test result.

In some embodiments, the visual result detection device is configured to capture, for each illumination period, a set of imaging data for each of a plurality of different sets of wavelengths. As a result, the techniques can generate a matrix of imaging information. For example, the techniques can generate an M×N matrix of information from M excitations and N photodiodes (e.g., where each photodiode captures light of different wavelength(s) or color(s)). As described herein, for example, the visual result detection device can be configured to capture color images with a white light illumination source and a color array with a Bayer array of red, green and blue filters, or a monochrome sensor and a wheel of red, green and blue filters. As another example, the techniques can use colored illumination sources (e.g., red, green and blue illumination sources) in sequence and use a monochrome sensor to capture the imaging data.

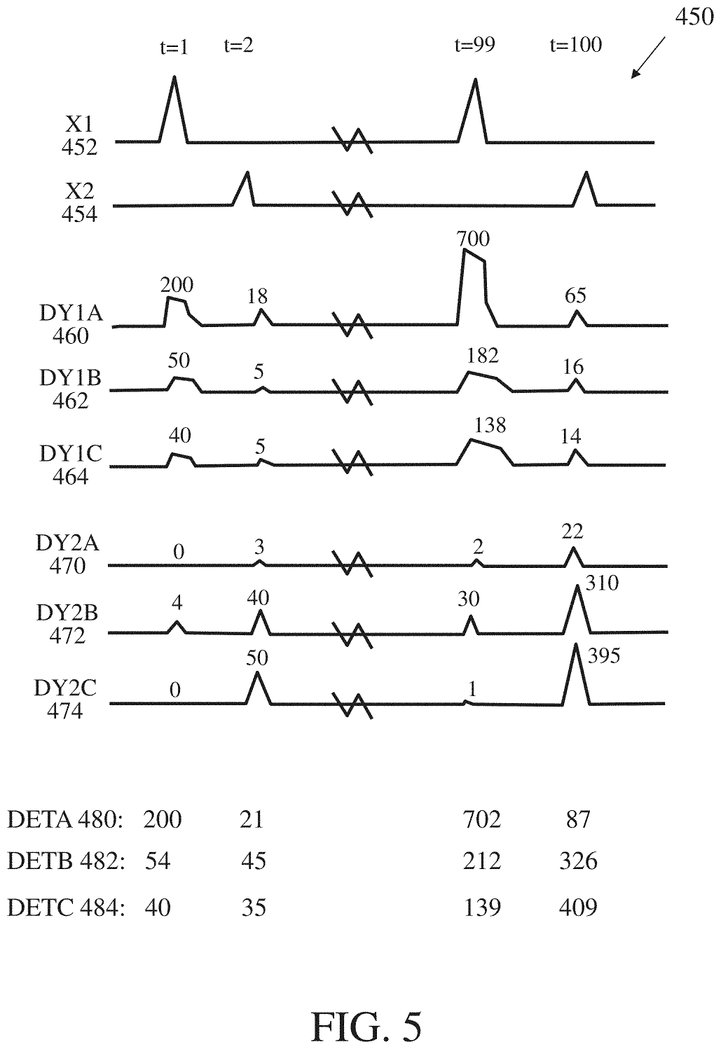

FIG. 5 is a diagram 500 showing a plurality of excitation illuminations and detection data from a plurality of light detectors, according to some embodiments. The diagram 500 shows excitation illumination X1 452 and X2 454. The diagram 500 also shows imaging data from three detectors A, B and C. In this example, the visual result detection device is configured to detect two different dyes DY1 and DY2 by exciting the dyes with different illumination wavelengths emitted by the illumination sources. Excitation illumination X1 452 emits illumination of a first wavelength (or first set of wavelengths) at times t=1 through t=99, in this example. Excitation illumination X2 454 emits illumination of a second wavelength (or second set of wavelengths) at times t=2 through t=100, in this example.

Referring further to FIG. 5, the emissions associated with the first dye DY1 that are captured by the detectors A, B and C are shown as DY1A 460, DY1B 462 and DY1C 464, respectively. The emissions associated with the second dye DY2 that are captured by the detectors A, B and C are shown as DY2A 470, DY2B 472 and DY2C 474, respectively. The detectors A, B and C capture the sum of the emissions of dyes DY1 and DY2, as shown by DETA 480, DETB 482 and DETC 484, respectively. As can be shown by the detected emissions of DY1 and DY2, excitation illumination X1 452 excites DY1 much more than DY2, while DY2 is almost only excited by excitation illumination X2 454.

As a result, effectively six signals (three associated with DY1 and three associated with DY2) can be followed over time, with illumination being largely independent for DY1 and DY2. As shown in this example, over time the detected emissions of both dyes DY1 and DY2 increase, which can be indicative of the presence and/or absence of one or more substances in the component, the occurrence (or lack thereof) of a reaction in the component, and/or the like. Various techniques can be used to analyze the received imaging data, such as one or more criteria specifying absolute values, sums, differences and/or ratios of imaging data (e.g., at one time instant t and/or over time).

In some embodiments, different detectors can be configured to be more sensitive to different emissions. In this example, detector A is more sensitive to dye 1 emissions. For example, for the emission at times t=1 and t=99, detector A has a stronger detection compared to detectors B and C. In this example, detector C is more sensitive to the emissions of dye 2. For example, for the emission at times t=2 and t=11, detector C has a stronger detection compared to detectors A and B.

In some embodiments, the visual result detection device can be configured combine the set of imaging data captured by different imaging devices for each illumination period. For example, at time t=1, the visual result detection device can be configured to determine a total detection time at t=1 by combining the detections DETA 480, DETB 482 and DET 484 at time t=1. In some embodiments, the visual result detection device can combine the different detections by computing a summation, by selecting a maximum, by computing an average, and/or the like.

The method 400 proceeds to act 408, and the visual result detection device determines, based on the image data, an aspect of the test. In some embodiments, the visual result detection device determines a test reading or a test result. In some embodiments, the visual result detection device determines one or more aspects of the test process (e.g., prior to the test providing a test reading or a test result), such as completion of a test step or a portion of a test step. For example, the visual result detection device can determine one or more aspects related to a sample preparation step, a heating step, a cooling step, a lysis step, a nucleic acid amplification step, and/or one or more other steps or portions of steps of the test. In some embodiments, the visual result detection device uses the image data to visually detect the presence or absence of one or more dyes in the component. For example, the techniques can detect the presence or absence of a set of one or more wavelengths and/or a set of wavelengths to determine whether or not one or more dyes are present or absent in a reaction tube. In some embodiments, the techniques can detect the presence or absence of a plurality of wavelengths and/or a plurality of sets of wavelengths. For example, different sets of wavelengths can be associated with different viruses, diseases, etc., and/or an invalid test. In an illustrative example, a first set of wavelengths can be associated with a positive test for COVID-19 and/or the influenza virus and/or the target nucleic acid, and a second set of wavelengths can be associated with an invalid test.

In some embodiments, the visual result detection device can use the detection (or absence) of one or more wavelengths and/or sets of wavelengths to determine a test reading or test result. For example, the visual result detection device can determine the presence or absence of COVID-19 and/or the influenza virus and/or a target nucleic acid based on whether a wavelength or set of wavelengths was detected in the image data. As another example, the visual result detection device can determine the test was not performed correctly (and therefore the test result is invalid) based on the detection of one or more wavelengths or sets of wavelengths. In some embodiments, the techniques can visually detect the presence (or absence) of one or more genes based on the presence or absence of one or more wavelengths or sets of wavelengths. For example, in some embodiments the visual result detection device detects the presence of a first gene associated with COVID-19 and a second gene associated with COVID-19. In some embodiments, the visual result detection device can be configured to visually detect a stage of a virus or disease. For example, in some embodiments the techniques can visually determine a stage of COVID-19 infection based on detecting the presence or absence of the first gene and the second gene. The stages can include, for example: an early COVID-19 infection, a late COVID-19 infection, a declining COVID-19 infection, and/or the like.

As described herein, in some embodiments the visual result detection device is programmed with and/or accesses test instructions. As described herein, the illumination and imaging process can be configured to capture a series of imaging data (e.g., as a matrix of imaging data from multiple sensors over time). The test instructions can include information on the dye behaviors when subject to illumination for various test results, such that the visual result detection device can determine a POSITIVE, NEGATIVE or INVALID result based on the imaging data without user intervention. For example, the test instructions can provide information that can be used to illuminate, image and/or analyze imaging data for a test for a certain virus using a certain type of chemistry. The test instructions can be updated for different viruses, different chemistry, and/or the like, in a manner that is transparent to the user. For example, when testing for the same virus but using a new/different generation chemistry (e.g., brighter dyes or probes, probes of different colors, faster amplification techniques, etc.), changes to the test instructions and/or new test instructions can be loaded and executed by the device. As another example, different test instructions can be used to test for different diseases.

In some embodiments, the techniques can include controlling aspects of the detectors and/or of the illumination sources. For example, a detector could have a programmable gain to achieve a dynamic range (e.g., more than otherwise provided by an ADC alone). As another example, different excitation illumination can be used with the system. For example, increasingly larger excitation to increase detection of very faint fluorescence, beyond the amplifier maximum gain. We have to be careful with bleaching, but that is also another operational info that can be conveyed by the NFC (max power to excite, how often, etc.)

### II. Computer Implementation

FIG. 6 is a diagram showing an illustrative implementation of a computer system 600 for visually detecting, via a reaction tube or other test component, presence or absence of a target (e.g. COVID-19 and/or an influenza virus and/or a target nucleic acid and/or other target pathogen), according to some embodiments. The computer system 600 may be used in connection with any of the embodiments of the technology described herein (e.g., such as the method of FIG. 3). The computer system 600 includes one or more processors 610 and one or more articles of manufacture that comprise non-transitory computer-readable storage media (e.g., memory 620 and one or more non-volatile storage media 630). The processor 610 may control writing data to and reading data from the memory 620 and the non-volatile storage device 630 in any suitable manner, as the aspects of the technology described herein are not limited in this respect. To perform any of the functionality described herein, the processor 610 may execute one or more processor-executable instructions stored in one or more non-transitory computer-readable storage media (e.g., the memory 620), which may serve as non-transitory computer-readable storage media storing processor-executable instructions for execution by the processor 610.

Computing device 600 may also include a network input/output (I/O) interface 640 via which the computing device may communicate with other computing devices (e.g., over a network), and may also include one or more user I/O interfaces 650, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Computing device 600 may also include a wireless module 660 (e.g., WiFi, Bluetooth, RFID, NFC, etc.), as described herein. In some embodiments, the computing device 600 can provide the imaging data to an application running on a remote computing device, such as a smartphone (e.g., which is configured to process the imaging data as described herein). Computing device 600 may further include one or more illumination detectors 670, as also described herein. Computing device 600 may also include one or more light sources 680, as described herein.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

The foregoing and following description of implementations provides illustration and description, but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

### III. Exemplary Tests for Use With the Color Reading Techniques

The following sections describe aspects of exemplary diagnostic devices, tests and test steps that can be used with the color reading techniques described herein, which are for illustrative purposes and are not intended to be limiting. Therefore, it should be appreciated that the color reading techniques described herein are not limited to such aspects, and can be used with any test, diagnostic device, or test kit.

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents, buffers, diluents, or any other appropriate materials may be contained within fluid containers (e.g., depots, reservoirs, receptacles, such as reaction tubes, cartridges, ad/or blister packs) of the device. In this way, the fluids and/or materials for the diagnostic test may be protected from contamination (either from surrounding gases/fluids or from cross-contamination within the device) until operation. The color reading techniques described herein can be used to monitor and/or detect colors of such reaction tubes, cartridges (e.g., wells of the cartridge), blister pack components, and/or the like.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. Thus, unlike diagnostic tests that require bulky equipment, diagnostic devices and systems described herein may be easily transported and/or easily stored in homes and businesses. In some embodiments, the diagnostic devices and systems may be relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time. In certain embodiments, for example, the housing including the one or more solutions may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the diagnostic device may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

A. Target Nucleic Acid Sequences

The diagnostic devices, systems, and methods, in some embodiments, may be used to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment. It should be appreciated that the techniques can be used to detect the presence or absence of any target, whether viral, bacterial, fungal, parasitic, protozoan, and/or the like.

B. Diagnostic Systems

According to some embodiments, diagnostic systems comprise a sample-collecting component (e.g., a swab) and a diagnostic device. In certain cases, the diagnostic device comprises a reaction tube, a cartridge (e.g., a microfluidic cartridge), and/or a blister pack. In some cases, the diagnostic device uses a visual test result detection device used to perform the color reading techniques described herein and/or comprises an additional detection component (e.g., a colorimetric assay). In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. Each of the one or more reagents may be in liquid form (e.g., in solution) or in solid form (e.g., lyophilized, dried, crystallized, air jetted). The diagnostic device may also comprise an integrated temperature control device (e.g., a heater), or the diagnostic system may comprise a separate temperature control device. In some embodiments, a heater may be a printed circuit board (PCB) heater that may be integrated into a diagnostic device.

C. Detection Components

In certain embodiments, the diagnostic device (e.g., reaction tube, cartridge, blister pack) comprises a detection component. As described herein, in some embodiments the detection components comprise a visual result detection device that is configured to visually sense and/or monitor a test (e.g., by monitoring for one or more quenched probes). In some embodiments, the detection component additionally and/or alternatively comprises a colorimetric assay. Examples are provided herein. In some embodiments, results of the colorimetric assay are read and/or analyzed by software (e.g., a mobile application). In some embodiments, the results of the colorimetric assay can be used in conjunction with and/or compared with the results determined by a visual result detection device.

In certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers (e.g., primers specific for one or more target nucleic acid sequences, primers specific for a positive test control, primers specific for a negative test control).

In operation, a sample may be deposited in the central sample chamber. In some cases, the sample may be combined with a reaction buffer in the central sample chamber. In certain cases, the central sample chamber may be heated and/or cooled to lyse cells within the sample. In some cases, the lysate may be directed to flow from the central sample chamber to the plurality of peripheral chambers comprising unique primers. In some cases, a colorimetric reaction may occur in each peripheral chamber, resulting in varying colors in the peripheral chambers. In some cases, the results within each peripheral chamber may be visible (e.g., through a clear film or other covering).

D. Detection Component for Use With Temperature Control Device

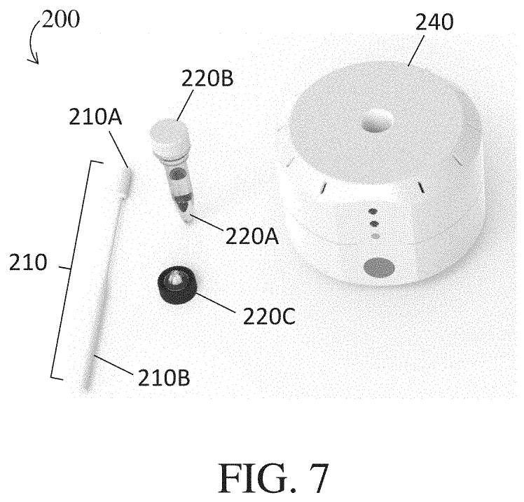

In some embodiments, a diagnostic device comprises a visual detection component that is configured for use with and/or incorporated as part of a temperature control device, such as a heater. One embodiment of an exemplary heater with a visual detection component is shown in FIG. 7. In FIG. 7, diagnostic system 200 comprises sample-collecting component 210, reaction tube 220, and temperature control device 240. As shown in FIG. 7, sample-collecting component 210 may be a swab comprising swab element 210A and stem element 210B. In certain embodiment, reaction tube 220 comprises tube 220A, first cap 220B, and second cap 220C, which can include one or more reagents (e.g., lysis reagents, nucleic acid amplification reagents, CRISPR/Cas detection reagents) and/or a reaction buffer.

In operation, a user may collect a sample that is inserted into the fluidic contents of tube 220A similar as described above. In some embodiments, reaction tube 220 may be inserted into temperature control device 240. Reaction tube 220 may be heated and/or cooled at one or more temperatures for one or more periods of time. Following heating and/or cooling, a visual detection component (not shown; incorporated within the heater 240) can perform a visual reading of the reaction tube 220. For example, the presence or absence of one or more target nucleic acid sequences may be determined by the visual detection component.

E. Cartridges

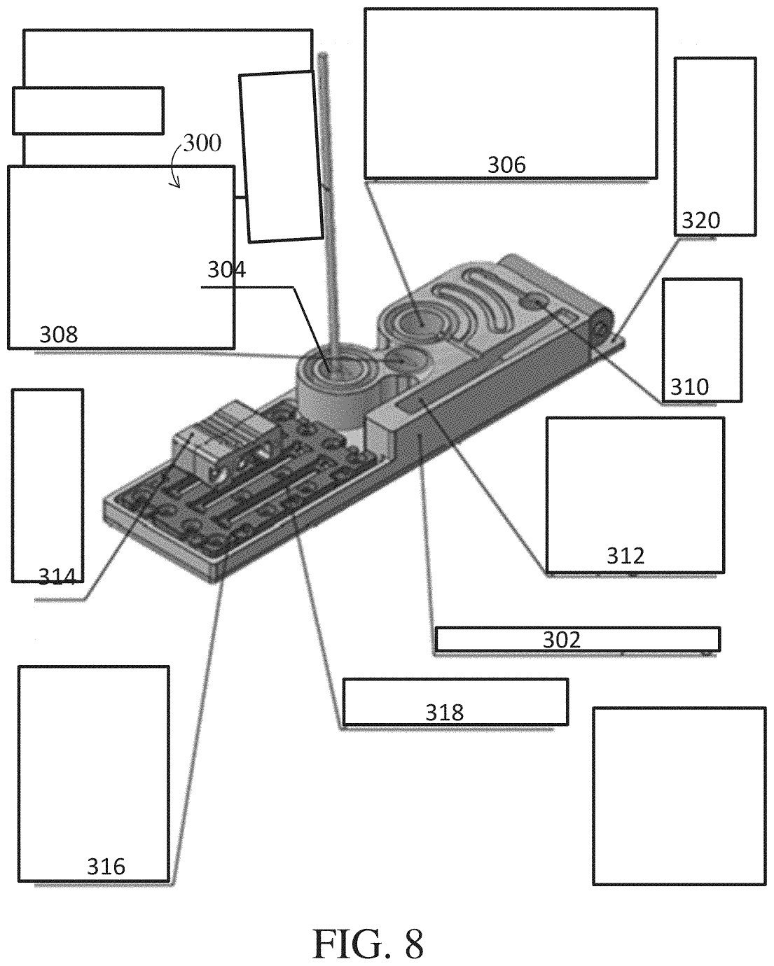

In some embodiments, a diagnostic device comprises a cartridge (e.g., a microfluidic cartridge). An exemplary cartridge is shown in FIG. 8, which includes a cartridge body 302 that comprises first reagent reservoir 304, second reagent reservoir 306, third reagent reservoir 308, vent path 310, and detection region 312. In some embodiments, detection region 312 comprises a visual test result detection component configured to detect one or more target nucleic acid sequences using the techniques described herein. For example, the visual result detection component can be configured to visually inspect a fluid in the detection region 312. In certain embodiments, the visual test result detection component is configured to detect one or more target nucleic acid sequences.

In some embodiments, cartridge 300 comprises an integrated heater 320. In some embodiments, heater 320 is a PCB heater. The PCB heater, in some embodiments, comprises a bonded PCB with a microcontroller, thermistors, and resistive heaters. In some embodiments, the heater comprises a USB- and/or battery-powered heater. In some embodiments, one or more heating elements of heater 320 may be in thermal communication with first reagent reservoir 304 and/or second reagent reservoir 306. In certain instances, for example, one or more heating elements of heater 320 are located under first reagent reservoir 304 and/or second reagent reservoir 306. In some cases, heater 320 runs a first heating protocol (e.g., a lysis heating protocol) and/or a second heating protocol (e.g., a nucleic acid amplification protocol). In some instances, heater 320 is pre-programmed to run the first heating protocol and/or the second heating protocol.

In operation, a user may use a swab to collect a sample from a subject (e.g., the user, a friend or family member of the user, or any other human or animal subject) and then expose the contents of first reagent reservoir 304. In some embodiments, chemical lysis may be performed by one or more lysis reagents (e.g., enzymes, detergents) in first reagent reservoir 304. In certain embodiments, thermal lysis may be performed by heater 320. In certain cases, for example, heater 320 may heat first reagent reservoir 304 according to a first heating protocol (e.g., a lysis heating protocol). In this manner, one or more cells within the sample may be lysed.

In some embodiments, the user may push pumping tool 314 along one or more pump lanes to transport at least a portion of the fluidic contents of first reagent reservoir 304 (e.g., comprising a lysate) to second reagent reservoir 306. In some instances, second reagent reservoir 306 comprises a second set of reagents (e.g., one or more nucleic acid amplification reagents). In certain cases, heater 320 may heat second reagent reservoir 306 according to a second heating protocol (e.g., a nucleic acid amplification heating protocol). In this manner, one or more target nucleic acid sequences may be amplified (if present within the sample).

In some embodiments, the fluidic contents of second reagent reservoir 306 (e.g., amplicon-containing fluid) may be transported to detection region 312 by pushing pumping tool 314 along one or more pump lanes. In this manner, at least a portion of the fluidic contents of second reagent reservoir 306 may be introduced into detection region 312. The visual test result detection component may be able to visually determine whether or not one or more target nucleic acid sequences are present based on the fluidic contents.

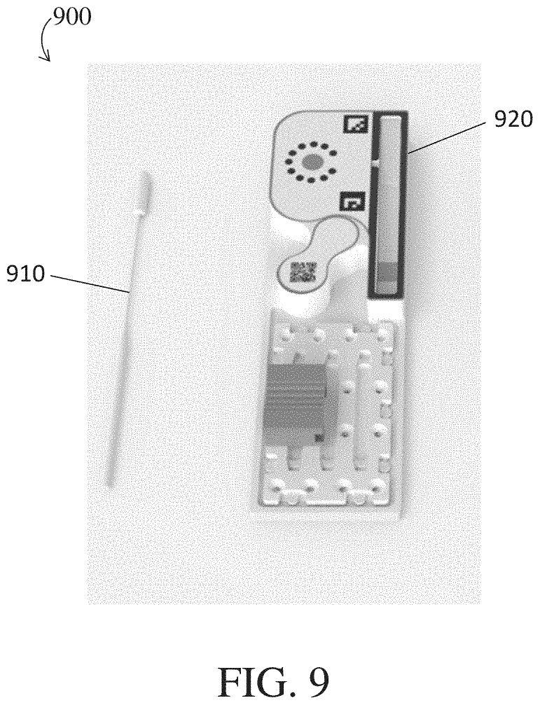

In some cases, a cartridge may be a component of a diagnostic system. For example, FIG. 9 illustrates an exemplary diagnostic system 900 comprising sample-collecting swab 910 and cartridge 920. In some embodiments, the diagnostic system may be used with an electronic device (e.g., a smartphone, a tablet) and associated software (e.g., a mobile application). In certain embodiments, for example, the software may provide instructions for using the cartridge, may read and/or analyze results, and/or report results. In certain instances, the electronic device may communicate with the cartridge (e.g., via a wireless connection).

F. Blister Pack Embodiments

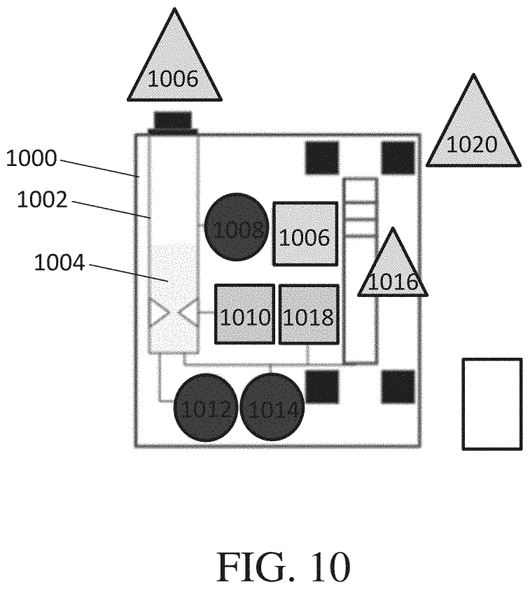

In some embodiments, a diagnostic device comprises one or more blister packs. One embodiment is shown in FIG. 10. In FIG. 10, diagnostic device 1000 comprises tube 1002 containing reaction buffer 1004. In certain embodiments, diagnostic device 1000 comprises a temperature control device in thermal communication with tube 1002.

In operation, a sample may be added through sample port 1006. A first blister pack 1008 comprising one or more lysis and/or decontamination reagents (e.g., UDG) are released from blister pack 1008 into tube 1002. In some embodiments, tube 1002 may be heated and/or cooled by a temperature control device (not shown in FIG. 10). In some cases, mechanism 1010 provides a physical mechanism to reduce sample volume as needed. In certain embodiments, one or more amplification reagents are released from amplification blister pack 1012 into tube 1002. In some instances, a dilution buffer may optionally be released from dilution blister pack 1014 into tube 1002. The sample is then flowed into detection region 1016, with mechanism 1018 ensuring that the sample accesses detection region 1016 at the appropriate time (e.g., after the processing is complete). A visual test result detection component may be able to visually determine a result of the test (e.g., whether or not one or more target nucleic acid sequences are present) based on the fluidic contents in the detection region 1016.

A further embodiment of the blister pack configuration comprises a swab in conjunction with a blister pack. A sample is taken using a swab. The swab is added to a tube comprising buffer and incubated for 10 minutes at room temperature. Then, a cap comprising one or more lysis reagents is added to the tube. Adding the cap dispenses the lysis reagents into the buffer and sample. The mixture is then heated at 95° C. for three minutes but the invention is not so limited. Other temperatures are envisioned. In some embodiments, the heating is accomplished with any heater described herein (e.g., boiling water, a fixed heat source). The reaction mixture is then allowed to cool for 1 minute, but this time period is not limiting as other time periods are envisioned. The resulting reaction mixture is then injected into a sample port of the blister pack (e.g., using a pipette). The cartridge is then sealed with seal tape and then shaken or otherwise agitated for 10 seconds but this time period is not limiting. The cartridge is heated for 20 minutes but this time period also is not limiting. In some embodiments, the cartridge is placed in a user's clothing pocket (e.g., back pocket of pants, front pocket of pants, front pocket of shirt) to heat the cartridge using the user's body heat. The user then pushes on a first blister to release a one or more amplification reagents (e.g., one or more reagents for LAMP, RPA, NEAR, or other isothermal amplification methods). The user presses on a second blister to release the dilution buffer and turns a valve to permit the mixture to proceed to a detection region after the appropriate amount of processing. The visual test result detection component can visually inspect the mixture to determine whether one or more target nucleic acid sequences are present in the sample.

G. Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g., mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

H. Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. In some embodiments, the one or more lysis reagents comprise one or more detergents. In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period.

I. Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). In some embodiments, DNA may be amplified according to any nucleic acid amplification method known in the art.

1. LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

2. RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

3. Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

J. Molecular Switches

As described herein, a sample undergoes lysis and amplification prior to detection. In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

K. Detection

In some embodiments, amplified nucleic acids (i.e., amplicons) may be detected using any suitable methods. As described herein, a visual test result detection component can be used to visually detect one or more target nucleic acid sequences by illuminating the test sample and monitoring for fluorescence of the sample in response to the illumination. In some embodiments, a colorimetric assay can be used in addition to the visual detection techniques. In some embodiments, the colorimetric assay can be provided as a second detection mechanism in addition to the visual test result detection component. For example, the colorimetric assay can be used to verify a test determination made by the visual test result detection component. As another example, the colorimetric assay can be used by the visual test result detection component as part of the data it uses to provide a test determination or test result.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

What is claimed is:

1. A rapid test method comprising:
performing an isothermal nucleic acid amplification-based rapid test;
accessing fluorescence data of a reaction tube of the test; and
visually detecting, via the fluorescence data, presence or absence of COVID-19 and/or an influenza virus and/or a target nucleic acid.

2. The rapid test method of claim 1, further comprising illuminating the reaction tube.

3. The rapid test method of claim 2, wherein illuminating the reaction tube further comprises illuminating the reaction tube at a plurality of different times.

4. The rapid test method of claim 2, wherein illuminating the reaction tube further comprises illuminating the reaction tube by a plurality of different illumination sources.

5. The rapid test method of claim 4, wherein illuminating the reaction tube further comprises illuminating the reaction tube by each of the plurality of different illumination sources at different times.

6. The rapid test method of claim 1, wherein visually detecting comprises visually detecting the presence or absence of one or more dyes in the reaction tube.

7. The rapid test method of claim 1, wherein visually detecting comprises visually detecting the presence or absence of a set of one or more wavelengths.

8. The rapid test method of claim 7, wherein visually detecting the presence or absence of the set of one or more wavelengths comprises visually detecting the presence or absence of a plurality of sets of wavelengths.

9. The rapid test method of claim 8, wherein the plurality of sets of wavelengths comprises:
a first set of wavelengths associated with a positive test for COVID-19 and/or the influenza virus and/or the target nucleic acid;
a second set of wavelengths associated with an invalid test; or some combination thereof.

10. The rapid test method of claim 7, wherein visually detecting the presence of COVID-19 and/or the influenza virus and/or the target nucleic acid comprises detecting the presence of the set of one or more wavelengths.

11. The rapid test method of claim 7, wherein visually detecting the absence of COVID-19 and/or the influenza virus and/or the target nucleic acid comprises detecting the absence of the set of one or more wavelengths.

12. The rapid test method of claim 7, further comprising detecting an invalid test.

13. The rapid test method of claim 12, wherein detecting the invalid test comprises detecting the presence of the set of one or more wavelengths.

14. The rapid test method of claim 1, wherein visually detecting, via the reaction tube, the presence or absence of COVID-19 comprises visually detecting a color of a solution in the reaction tube indicative of the presence or absence of:
a first gene associated with COVID-19; and
a second gene associated with COVID-19.

15. The rapid test method of claim 14, further comprising determining a stage of COVID-19 infection based on detecting the presence or absence of the first gene and the second gene.

16. The rapid test method of claim 15, wherein determining the stage comprises determining an early COVID-19 infection, a late COVID-19 infection, a declining COVID-19 infection, or some combination thereof.

17. An apparatus comprising:
at least one computer hardware processor; and
at least one non-transitory computer-readable storage medium storing processor executable instructions that, when executed by the at least one computer hardware processor, cause the at least one computer hardware processor to perform:
accessing fluorescence data of a reaction tube of a test; and
visually detecting, via the fluorescence data, presence or absence of COVID-19 and/or an influenza virus and/or a target nucleic acid.

18. The apparatus of claim 17, further comprising an illumination source in electrical communication with the at least one computer hardware processor, wherein the illumination source is configured to illuminate the reaction tube.

19. The apparatus of any of claim 18, wherein the illumination source comprises a programmable current illumination source.

20. The apparatus of any of claim 19, wherein the illumination source comprises a constant current illumination source.

21. The apparatus of any of claim 20, wherein the illumination source comprises a light emitting diode or a laser diode.

22. The apparatus of claim 20, further comprising a light detector configured to capture fluorescence data of the reaction tube of the test.

23. The apparatus of claim 22, wherein the light detector comprises at least one a photodetector, a photodiode, a camera, a transimpedance amplifier, or a filter.
